(19) United States
(12) Patent Application Publication
(10) Pub. No.: US 2018/0299428 A1
(43) Pub. Date: Oct. 18, 2018

(54) METHOD FOR PROGNOSING ADVERSE CARDIOVASCULAR OUTCOME IN PATIENTS WITH CORONARY ARTERY DISEASES

(71) Applicants: TIFFANY WU, NEW YORK, NY (US); Chi-Hua Foundation, New Taipei City (TW)

(72) Inventor: WAN-LIN WU, New Taipei City (TW)

(21) Appl. No.: 15/951,893

(22) Filed: Apr. 12, 2018

Related U.S. Application Data
(60) Provisional application No. 62/485,371, filed on Apr. 13, 2017.

(51) Int. Cl. G01N 33/50; C12Q 1/6809

(52) U.S. Cl. CPC G01N 33/5023; C12Q 1/6809; G01N 2800/50; G01N 2800/325; G01N 2800/56; C12Q 2600/158

(57) ABSTRACT

A method for prognosing adverse cardiovascular events or deaths in patients with coronary artery diseases, includes the steps of selecting at least one target long non-coding RNA from the group comprising lnc-CXXC11-1:2, lnc-CCT7-1:1, LINC00930:1, TSC22D1-AS1:3, lnc-MEGF10-6:1, lnc-INA-1:1, HPN-AS1:2, lnc-TFAP4-3:1, lnc-SCN8A-2:2, lnc-NCF1-1, BRE-AS1, lnc-ZFAT-6, lnc-SLC46A3-5, lnc-CXCL3-2, lnc-AL137798.1-8, lnc-BCL2L2-PABPN1-1, and lnc-EBF3-4, analyzing the at least one target long non-coding RNA in a sample from a patients with coronary artery diseases, and utilizing the expression as a predictor for adverse cardiovascular events or deaths in patients with coronary artery diseases.

### CROSS-REFERENCE

The present application claims the priority the provisional application No. 62/485,371 field on Apr. 13, 2017.

### BACKGROUND OF THE INVENTION

### Field of the Invention

The present invention relates to a method for prognosing clinical ischemia in patients with coronary artery diseases, especially utilizing expression of specific long non-coding RNAs in patients with coronary artery diseases to predict whether the patients belongs to a high-risk group to have cardiovascular ischemia.

### Description of the Related Art

Coronary artery disease, a.k.a. CAD, is a disease which is commonly induced by coronary arterial atherosclerosis. It is due to that cholesterol or fat accumulates in inner wall of arterial vasculature to form atherothrombosis, and sudden blood clot formation in coronary artery may cause ischemia, chest pain, tachypnea, pain in upper limb, perspire, vomit, and the like. If the arterial vasculature is fully clogged by blood clots, the oxygen cannot reach the heart muscle, further cause the myocardial injury. Coronary artery diseases may have various clinical manifestations, such as stable angina pectoris, acute coronary syndrome (unstable angina pectoris or myocardial infarction), asymptomatic myocardial ischemia, ischemic cardiomyopathy and sudden cardiac deaths, and the like.

Currently, the methods to diagnose coronary artery diseases include: electrocardiogram, echocardiography, heart tomography and biomarkers such as cardiac enzymes. Currently there lacks a sensitive and reliable biomarker to determine the risk of adverse cardiovascular (CV) events or death in CAD patients.

Dyslipidemia may also cause atherosclerosis and be one of the reason to induce many cardiovascular diseases. Because the diet habit changes nowadays, the increased eating-out and fast food may cause more absorption of fat and more atherosclerosis. Currently, there is no clinical reliable biomarker to test atherosclerosis for patients with dyslipidemia, and there in no useful prognosis-evaluating system to evaluate the risk of the patients with atherosclerosis to have adverse CV events or deaths in the future.

Long Non-Coding RNAs (lncRNAs) are a class of RNA transcripts longer than 200 bp that are not translated into proteins. In recent years, studies have suggested that lncRNAs are involved in the regulation of cellular function, and lncRNAs function critically in regulating gene expression, maintaining genome integrity, compensating gene dosage, genome imprinting, mRNA processing, and cell differentiation and development. Aberrantly expressed lncRNAs contribute to the development of many diseases including cancers, immune diseases and neurological disorders.

### SUMMARY OF THE INVENTION

The purpose of the present invention is to provide a method for prognosing clinical ischemia in patients with coronary artery diseases. It utilizes detecting a change of the expression of a specific long non-coding RNA in the patients with coronary artery diseases, to prognose the possibility of adverse CV events and deaths in the patients with coronary artery diseases in the future.

To achieve the aforementioned purpose, the technical feature is analyzing the expression of at least one target long non-coding RNA in patients with coronary artery diseases. The target long non-coding RNA is selected from nc-CXXC11-1:2, lnc-CCT7-1:1, LINC00930:1, TSC22D1-AS1:3, lnc-MEGF10-6:1, lnc-INA-1:1, HPN-AS1:2, lnc-TFAP4-3:1, lnc-SCN8A-2:2, lnc-NCF1-1, BRE-AS1, lnc-ZFAT-6, lnc-SLC46A3-5, lnc-CXCL3-2, lnc-AL137798.1-8, lnc-BCL2L2-PABPN1-1, lnc-EBF3-4 and any combination thereof. When the expression of the target long non-coding RNA of the patients is higher than a default value, the patients belongs to the high-risk group of having adverse CV events or deaths in the future. Furthermore, it provides doctors with potential treatments and preps for the patients and their family.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The detailed description of the drawings particularly refers to the accompanying figures in which:

FIG. 1 shows a comparison of expressions of IncRNAs.

FIG. 2 shows a ROC analysis of target lncRNAs, lnc-CXXC11-1:2 and lnc-CCT7-1:1.

FIG. 3 shows a ROC analysis of target lncRNAs, lnc-NCF1-1, BRE-AS1, lnc-ZFAT-6 and lnc-SLC46A3-5.

FIG. 4 shows a ROC analysis of target lncRNAs, lnc-CXCL3-2, lnc-BCL2L2-PABPN1-1, lnc-Al137798.1-8 and lnc-EBF3-4.

FIG. 5 shows a Kaplan-Meier survival analysis of target lncRNAs, lnc-CXXC11-1:2 and lnc-CCT7-1:1.

FIG. 6 shows a Kaplan-Meier survival analysis of target lncRNAs, lnc-NCF1-1, BRE-AS1, lnc-ZFAT-6 and lnc-SLC46A3-5.

FIG. 7 shows a Kaplan-Meier survival analysis of target lncRNAs, lnc-CXCL3-2, lnc-BCL2L2-PABPN1-1, lnc-Al137798.1-8 and lnc-EBF3-4.

FIG. 8 shows the expression of target lncRNAs in cell lines of cardiomyopathy.

FIG. 9 shows the expression of target lncRNAs in cell lines of cardiovascular and cardiac fibers.

### DETAILED DESCRIPTION OF THE INVENTION

For a better knowledge and understanding of the present disclosure as a courtesy for the examiner, the technical features and process of the present disclosure have been illustrated by the embodiments and drawings below.

The present embodiment analyzed the difference of expression of long non-coding RNAs between CAD patients with adverse cardiovascular (CV) events or deaths and CAD patients without any adverse cardiovascular events or deaths, in order to find the biomarkers for detection.

The sample type used in the present embodiment is plasma from blood samples.

No CV Event Group: After following up for five years, plasma samples from CAD patients without any adverse CV events or deaths.

With CV Event Group: After following up for five years: plasma samples from CAD patients with adverse CV events or deaths.

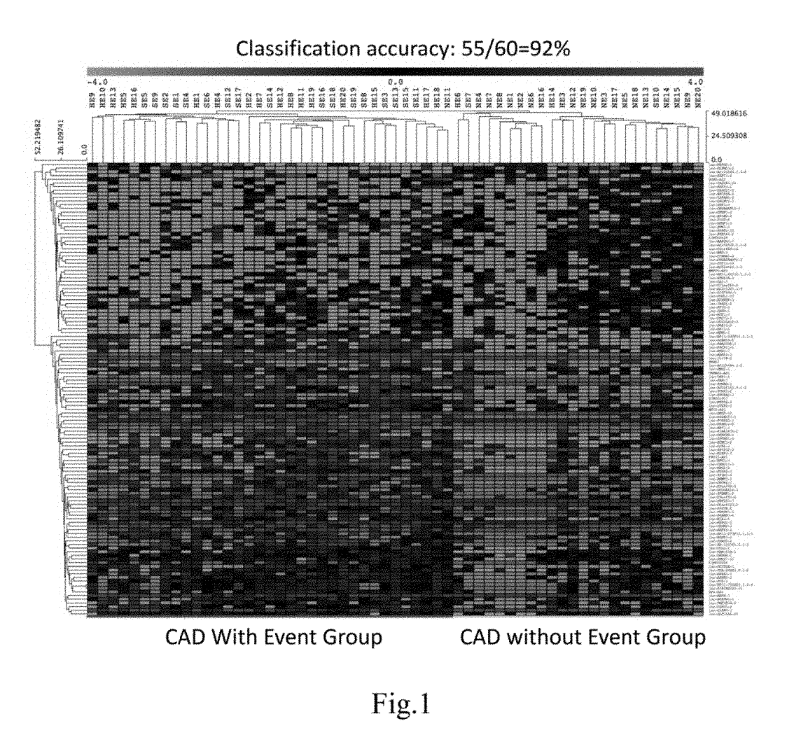

First, the types and expression of lncRNAs from two sample groups were analyzed by Next-generation sequencing. The results were shown in FIG. 1. The expressions of lncRNA from nine plasma samples from ‘With CV Event Group’ were higher than the other samples from ‘No CV Event Group’.

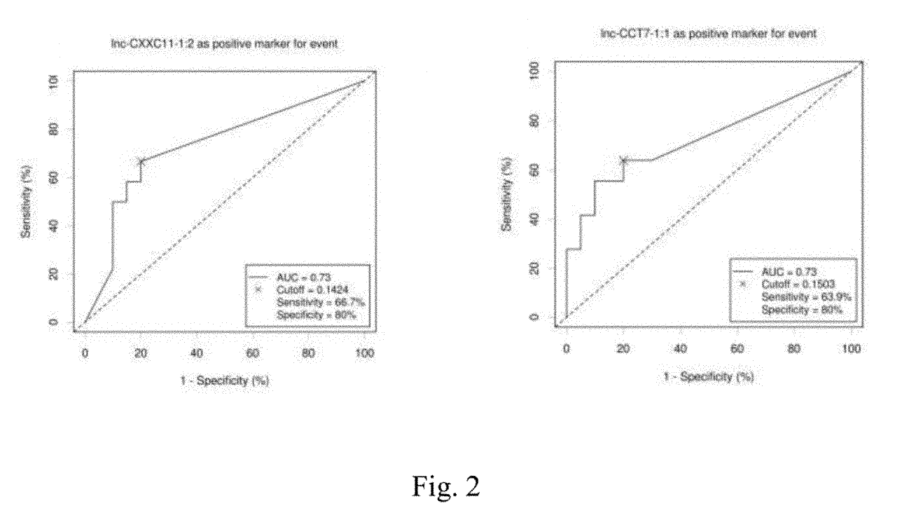

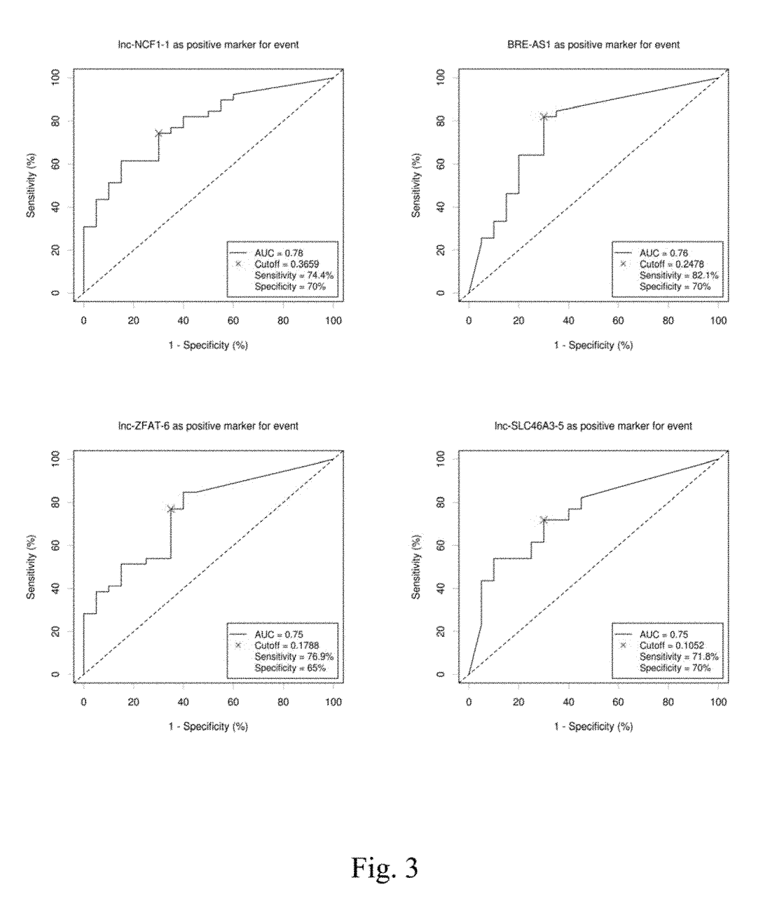

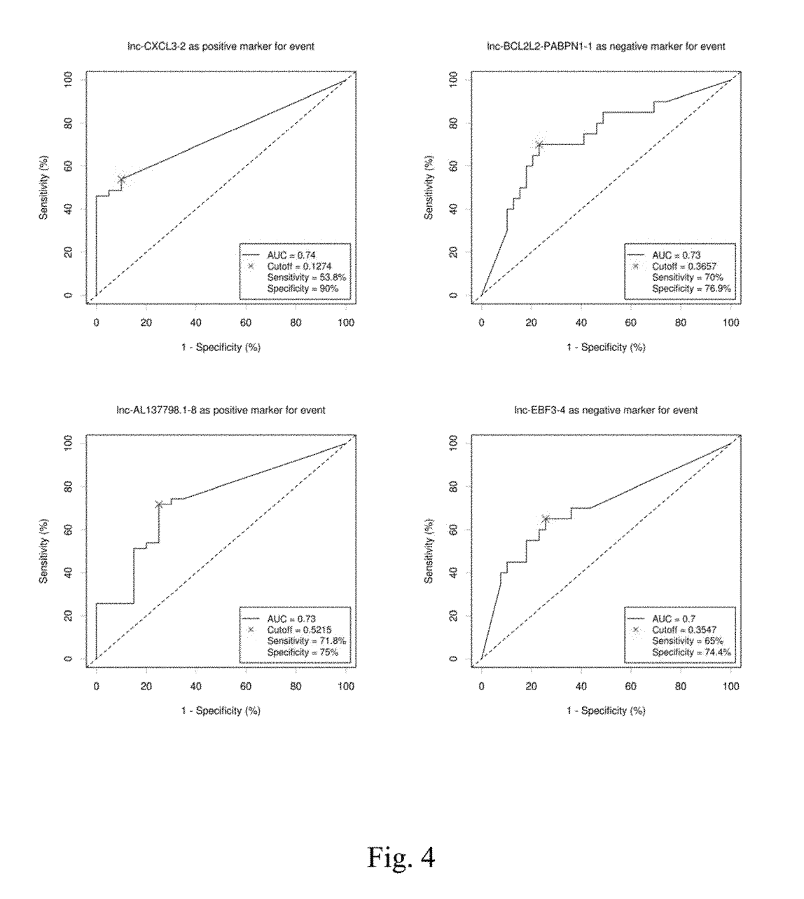

Furthermore, FIG. 2 to FIG. 4 shows the area under ROC curve of these target lncRNAs. The area under the ROC curve, a.k.a. area under curve, AUC, Hazard Ratio analyses were conducted. The analysis report was shown in Table 1, wherein under the assigned cutoff value for the nine lncRNAs, AUC for all these nine samples was higher than 0.65. The expressions of lnc-CXXC11-1:2, lnc-CCT7-1:1, TSC22D1-AS1:3, lnc-INA-1:1, lnc-TFAP4-3:1, lnc-SCN8A-2:2, lnc-NCF1-1, BRE-AS1, lnc-ZFAT-6, lnc-SLC46A3-5, lnc-CXCL3-2 and lnc-AL137798.1-8 from CAD patients with adverse CV events or deaths were higher than those from CAD patients without any adverse CV events or deaths.

They belong to positive biomarkers. The expressions of 1 LINC00930:1, lnc-MEGF10-6:1, HPN-AS1:2, lnc-BCL2L2-PABPN1-1 and lnc-EBF3-4 were lower than those from CAD patients without any adverse CV events or deaths. They belong to negative biomarkers.

TABLE 1
Plasma lncRNAs that are significant predictors for
adverse cardiovascular events or deaths in CAD patients

| | RPKM | ROC analysis | Hazard Ratio | |
|---|---|---|---|---|
| Plasma lncRNA | Cut-off | (AUC, 95% CI) | (HR, 95% CI) | P value |
| lnc-CXXC11-1:2 | 0.1424 | 0.73 (0.60-0.86) | 3.18 (1.56-6.47) | <0.0001 |
| lnc-CCT7-1:1 | 0.1503 | 0.73 (0.60-0.84) | 3.70 (1.83-7.49) | 0.0001 |
| LINC00930:1 | 0.1159 | 0.66 (0.50-0.83) | 0.27 (0.12-0.61) | 0.0007 |
| TSC22D1-AS1:3 | 0.1858 | 0.74 (0.61-0.87) | 2.68 (1.31-5.50) | 0.0051 |
| lnc-MEGF10-6:1 | 0.4372 | 0.67 (0.53-0.82) | 0.36 (0.17-0.78) | 0.0066 |
| lnc-INA-1:1 | 0.3043 | 0.69 (0.56-0.81) | 2.46 (1.25-4.84) | 0.0071 |
| HPN-AS1:2 | 0.4270 | 0.70 (0.54-0.85) | 0.37 (0.17-0.79) | 0.0076 |
| lnc-TFAP4-3:1 | 0.2327 | 0.70 (0.57-0.83) | 2.13 (1.07-4.24) | 0.0280 |
| lnc-SCN8A-2:2 | 0.2449 | 0.70 (0.57-0.83) | 2.16 (1.05-4.42) | 0.0320 |
| lnc-NCF1-1 | 0.3659 | 0.78 | 3.01 (1.46-6.24) | 0.002 |
| BRE-AS1 | 0.2478 | 0.76 | 3.96 (1.73-9.06) | <0.001 |
| lnc-ZFAT-6 | 0.1788 | 0.75 | 3.35 (1.58-7.09) | 0.001 |
| lnc-SLC46A3-5 | 0.1052 | 0.75 | 3.14 (1.55-6.35) | 0.001 |
| lnc-CXCL3-2 | 0.1274 | 0.74 | 3.26 (1.70-6.26) | <0.001 |
| lnc-BCL2L2- | 0.2478 | 0.73 | 0.29 (0.14-0.62) | 0.007 |
| PABP N1-1 | | | | |
| lnc-AL137798.1-8 | 0.5215 | 0.73 | 3.08 (1.52-6.25) | 0.001 |
| lnc-EBF3-4 | 0.3547 | 0.70 | 0.38 (0.18-0.79) | 0.007 |

Survival Test

The present embodiment observed the progression of the disease in CAD patients and the expressions of lncRNAs in vivo periodically over time, and conducted the Kaplan-Meier survival analysis.

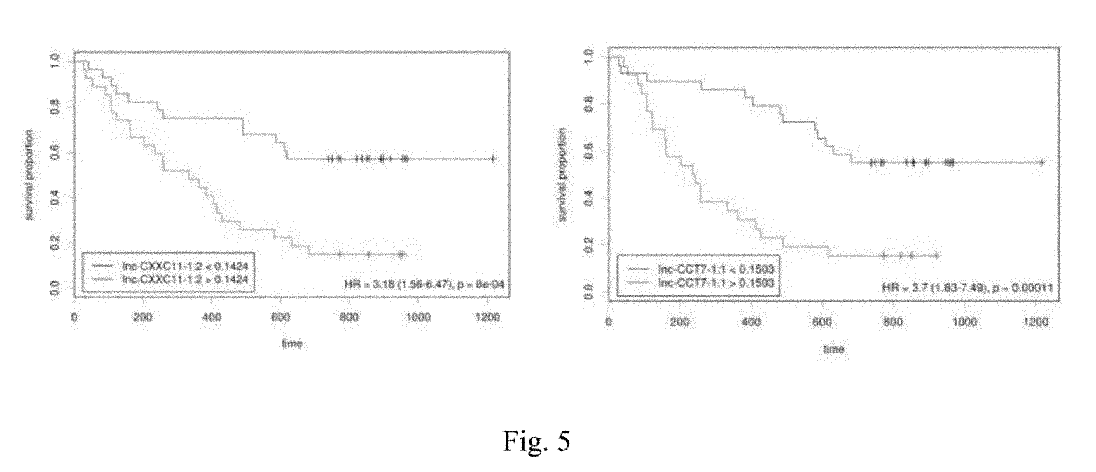

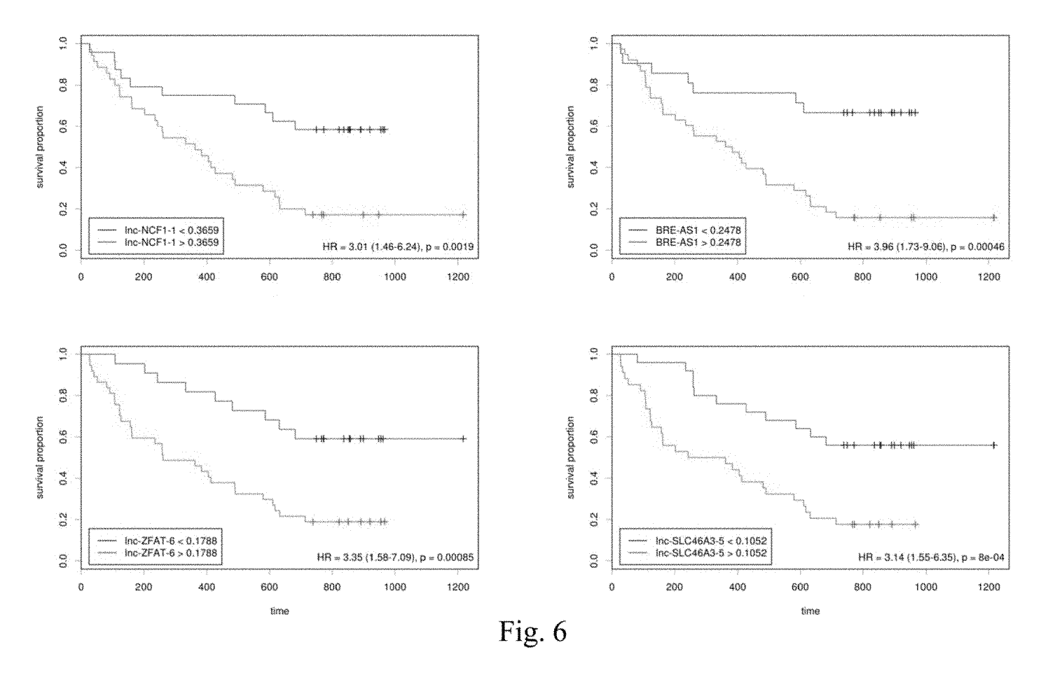

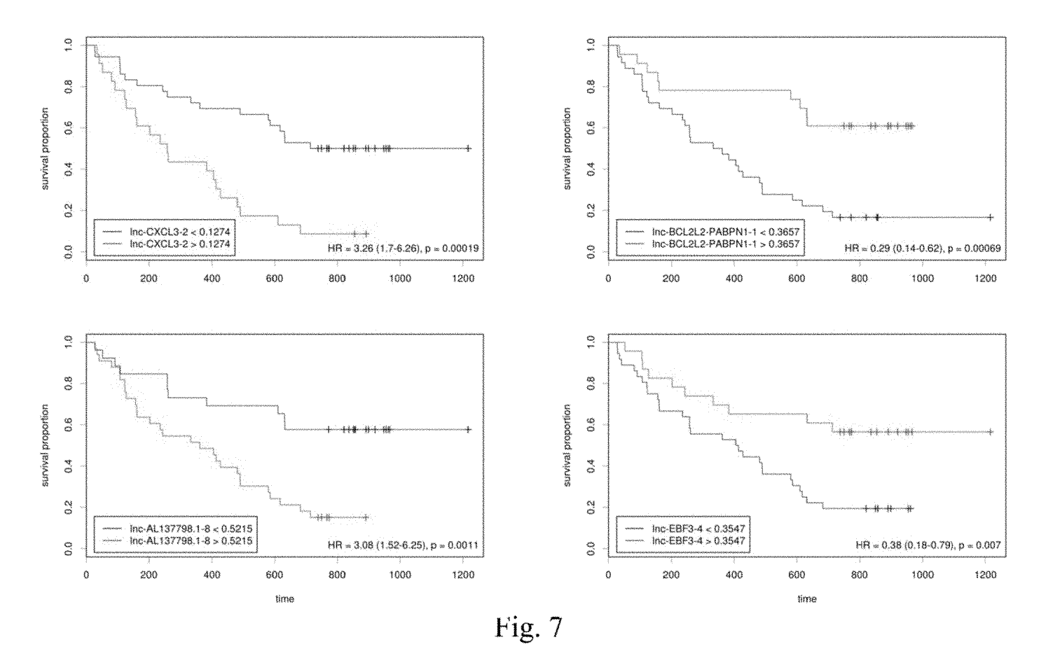

Please refer to FIG. 5 to FIG. 7, after the long-term following-up, when the expressions of the target lncRNAs belonging to these positive biomarkers in CAD patients were higher than the RPKM cut-off value, the adverse CV events were found in the subjects. The survival rate decreased in Kaplan-Meier survival analysis significantly. When the expressions of the target lncRNAs as a negative biomarker were lower than the RPKM cut-off value, the adverse CV events were also found from the subjects, and the survival rate decreased by Kaplan-Meier survival analysis significantly.

For example, please refer to FIG. 5, after the long-term following-up, when the expressions of lnc-CXXC11-1:2 and lnc-CCT7-1:1 in the CAD patients were higher than the RPKM cut-off value, the adverse CV events or deaths were found in the subjects. The survival rate decreased in Kaplan-Meier survival analysis significantly. When the expressions of the target lncRNAs as a negative biomarker were lower than the RPKM cut-off value, the adverse CV events or deaths were also found in the subjects, and the survival rate decreased in Kaplan-Meier survival analysis significantly. Whereas, when the expressions from of lnc-CXXC11-1:2 and lnc-CCT7-1:1 from the CAD patients were lower than RPKM cut-off value, the physical conditions of the subjects remained stable.

### Gene Expression Experiment 1

The present embodiment analyzed the expressions of lnc-CXXC11-1:2 and lnc-CCT7-1:1 of target lncRNAs from left ventricular (LV) tissues, in which the samples of ischemic cardiomyopathy (ICM), non-ischemic cardiomyopathy (NICM) and non-failing hearts were used for analysis.

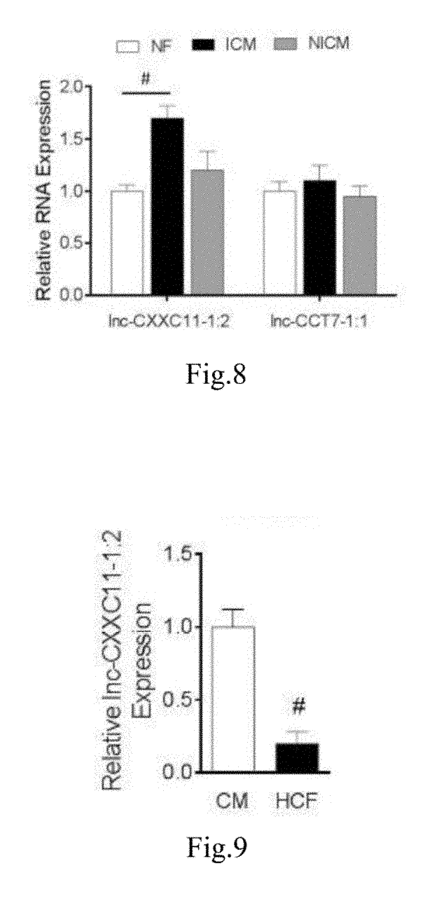

Please refer to FIG. 8, the expressions of ICM samples, both lnc-CXXC11-1:2 and lnc-CCT7-1:1, are higher than the other two samples.

### Gene Expression Experiment 2

The present experiment analyzed the expression of lnc-CXXC11-1:2 in human cardiomyocytes (CM) and cardiac fibroblasts (HCF).

FIG. 9 indicates that the expression of lnc-CXXC11-1:2 in human cardiomyocytes is higher than in cardiac fibroblasts.

Therefore, when the target lncRNAs of the present embodiment expressed in cells related to cardiovascular diseases, it is relevant to adverse CV events.

To summary the aforementioned embodiments, the expressions of the target lncRNAs in CAD patients found in the present embodiments relate to adverse cardiovascular events or deaths. When one or more expressions of the target lncRNAs in a CAD patients is higher or lower than the RPKM cut-off value, the CAD patients would be considered as a high-risk group to have adverse cardiovascular events or deaths in the future. Furthermore, one or any combination of the target lncRNAs of the present disclosure may be used for prognosis. That is being said that the present method may be applied to the clinic tests or treatments.

The comparison and test of these target lncRNAs in the subjects and RPKM cut-off value have to consider whether these target lncRNAs are positive biomarkers or negative biomarkers.

The method to test the expressions of lncRNAs include but not limited to: reverse transcriptase-polymerase chain reaction (RT-PCR), quantitative real-time PCR (qPCR), digital droplet PCR (ddPCR), microarray, serial analysis of gene expression (SAGE), next-generation RNA sequencing, massively parallel signature sequencing (MPSS), ELISA, in situ hybridization (ISH), mass spectrometry (MS), RNA pull-down, single nucleotide polymorphisms (SNPs), and the like.

What is claimed is:

1. A method for prognosing adverse cardiovascular outcome in patients with coronary artery diseases, comprising:
determining an expression of at least one target long non-coding RNA, lncRNA, in a blood sample from a patients with coronary artery diseases, wherein the target lncRNA is selected from lnc-CXXC11-1:2, lnc-CCT7-1:1, LINC00930:1, TSC22D1-AS1:3, lnc-MEGF10-6:1, lnc-INA-1:1, HPN-AS1:2, lnc-TFAP4-3:1, lnc-SCN8A-2:2, lnc-NCF1-1, BRE-AS1, lnc-ZFAT-6, lnc-SLC46A3-5, lnc-CXCL3-2, lnc-AL137798.1-8, lnc-BCL2L2-PABPN1-1, lnc-EBF3-4 and any combination thereof; and
comparing the expression of the target lncRNA in the blood sample from the patients with coronary artery diseases to a default value, wherein the comparison highly relates to a high-risk group of having adverse cardiovascular events or deaths for the patients with coronary artery diseases.

2. The method of claim 1, wherein the patients belongs to the high-risk group of having adverse cardiovascular events or deaths, when the expression of the target lncRNA of the patients is higher than the default value, and the target lncRNAs include: lnc-CXXC11-1:2, lnc-CCT7-1:1, TSC22D1-AS1:3, lnc-INA-1:1, lnc-TFAP4-3:1, lnc-SCN8A-2:2, lnc-NCF1-1, BRE-AS1, lnc-ZFAT-6, lnc-SLC46A3-5, lnc-CXCL3-2, and lnc-AL137798.1-8.

3. The method of claim 1, wherein the patients belongs to the high-risk group of having adverse cardiovascular events or deaths, when the expression of the target lncRNA of the patients is lower than the default value, and the target lncRNAs include: LINC00930:1, lnc-MEGF10-6:1, HPN-AS1:2, lnc-BCL2L2-PABPN1-1 and lnc-EBF3-4.

4. The method of claim 1, wherein the default value is calculated by comparing the expressions of the target lncRNAs from a blood sample of the CAD patients with adverse cardiovascular events or deaths and a blood sample of the CAD patients without any adverse cardiovascular events or deaths, to generate a RPKM cutoff value.

5. The method of claim 2, wherein the default value is calculated by comparing the expressions of the target lncRNAs from a blood sample of the CAD patients with adverse cardiovascular events or deaths and a blood sample of the CAD patients without any adverse cardiovascular events or deaths, to generate a RPKM cutoff value.

6. The method of claim 3, wherein the default value is calculated by comparing the expressions of the target lncRNAs from a blood sample of the CAD patients with adverse cardiovascular events or deaths and a blood sample of the CAD patients without any adverse cardiovascular events or deaths, to generate a RPKM cutoff value.

7. The method of claim 1, wherein the expression of the target lncRNA is quantified by one of the following methods: Next-generation sequencing, reverse transcriptase-polymerase chain reaction (RT-PCR), quantitative real-time PCR (qPCR), digital droplet PCR (ddPCR), microarray, serial analysis of gene expression (SAGE), next-generation RNA sequencing, massively parallel signature sequencing (MPSS), ELISA, in situ hybridization (ISH), mass spectrometry (MS), RNA pull-down and single nucleotide polymorphisms (SNPs).

8. The method of claim 2, wherein the expression of the target lncRNA is quantified by one of the following methods: Next-generation sequencing, reverse transcriptase-polymerase chain reaction (RT-PCR), quantitative real-time PCR (qPCR), digital droplet PCR (ddPCR), microarray, serial analysis of gene expression (SAGE), next-generation RNA sequencing, massively parallel signature sequencing (MPSS), ELISA, in situ hybridization (ISH), mass spectrometry (MS), RNA pull-down and single nucleotide polymorphisms (SNPs).

9. The method of claim 3, wherein the expression of the target lncRNA is quantified by one of the following methods: Next-generation sequencing, reverse transcriptase-polymerase chain reaction (RT-PCR), quantitative real-time PCR (qPCR), digital droplet PCR (ddPCR), microarray, serial analysis of gene expression (SAGE), next-generation RNA sequencing, massively parallel signature sequencing (MPSS), ELISA, in situ hybridization (ISH), mass spectrometry (MS), RNA pull-down and single nucleotide polymorphisms (SNPs).

10. The method of claim 4, wherein the expression of the target lncRNA is quantified by one of the following methods: Next-generation sequencing, reverse transcriptase-polymerase chain reaction (RT-PCR), quantitative real-time PCR (qPCR), digital droplet PCR (ddPCR), microarray, serial analysis of gene expression (SAGE), next-generation RNA sequencing, massively parallel signature sequencing (MPSS), ELISA, in situ hybridization (ISH), mass spectrometry (MS), RNA pull-down and single nucleotide polymorphisms (SNPs).

11. The method of claim 5, wherein the expression of the target lncRNA is quantified by one of the following methods: Next-generation sequencing, reverse transcriptase-polymerase chain reaction (RT-PCR), quantitative real-time PCR (qPCR), digital droplet PCR (ddPCR), microarray, serial analysis of gene expression (SAGE), next-generation RNA sequencing, massively parallel signature sequencing (MPSS), ELISA, in situ hybridization (ISH), mass spectrometry (MS), RNA pull-down and single nucleotide polymorphisms (SNPs).

12. The method of claim 6, wherein the expression of the target lncRNA is quantified by one of the following methods: Next-generation sequencing, reverse transcriptase-polymerase chain reaction (RT-PCR), quantitative real-time PCR (qPCR), digital droplet PCR (ddPCR), microarray, serial analysis of gene expression (SAGE), next-generation RNA sequencing, massively parallel signature sequencing (MPSS), ELISA, in situ hybridization (ISH), mass spectrometry (MS), RNA pull-down and single nucleotide polymorphisms (SNPs).
